(19) United States
(12) Patent Application Publication
(10) Pub. No.: US 2021/0167304 A1
(43) Pub. Date: Jun. 3, 2021

(54) NEW EMITTER MATERIALS AND MATRIX MATERIALS FOR OPTOELECTRONIC AND ELECTRONIC COMPONENTS, IN PARTICULAR ORGANIC LIGHT-EMITTING DIODES (OLEDS)

(71) Applicant: Technische Universität Dresden, Dresden (DE)

(72) Inventors: Reinhard Scholz, Freital (DE); Ramunas Lygaitis, Dresden (DE); Paul Kleine, Dresden (DE); Simone Lenk, Dresden (DE); Sebastian Reineke, Dresden (DE)

(21) Appl. No.: 16/065,305

(22) PCT Filed: Dec. 28, 2016
(86) PCT No.: PCT/EP2016/082763

§ 371 Date: Aug. 2, 2018

(30) Foreign Application Priority Data
Dec. 28, 2015 (DE) 10 2015 122 869.6

(51) Int. Cl. H01L 51/00; C09K 11/06; C07F 7/08; C07D 279/34; C07D 417/14

(52) U.S. Cl. CPC H01L 51/0094; C09K 11/06; C07F 7/0816; C07D 279/34; H01L 51/5012; C07D 417/14; H01L 51/0072; H01L 51/0074; C09K 2211/1018; H01L 51/0071

(57) ABSTRACT

The invention relates to compounds, comprising at least one donor group and at least one acceptor group, in which the transition energy of the lowest excited triplet hack into the ground state both of the corresponding donor molecule and of the corresponding acceptor molecule being at least 2.2 eV, and the use thereof as an emitter or a carrier material in an optoelectronic component.

The present invention provides compounds as described herein and for the use thereof as emitter or host material in an optoelectronic component.

The development of novel functional compounds for use in electronic devices is currently the subject of intensive research. The aim here is the development and study of compounds which have not been used to date in electronic devices, and the development of compounds which enable an improved profile of properties of the devices.

According to the present invention, the term “optoelectronic component” is understood to mean inter aria organic integrated circuits (OICs), organic field-effect transistors (OFETs), organic thin-film transistors (OTFTs), organic light-emitting transistors (OLETs), organic solar cells (OSCs), organic optical detectors, organic photoreceptors, organic field-quench devices (OFQDs), organic light-emitting electrochemical cells (OLECs), organic laser diodes (O-laser) and organic electrolurninescent devices such as organic light-emitting diodes (OLEDs).

The construction of organic electroluminescent devices (OLEDs), in which the compounds described herein can preferably be used as functional materials, is known to those skilled in the art and is described inter alia in patent publications U.S. Pat. Nos. 4,539,507, 5151629, EP 0676461 and WO 1998/27136.

In relation to the performance data of the OLEDs, especially with regard to broad commercial use, further improvements are still required. Of particular significance in this connection are the lifetime, efficiency and operating voltage of the OLEDs, and the color values achieved. More particularly, there is an urgent requirement in the industry for long-lived, efficient blue emitters for OLEDs which are additionally also still producible inexpensively.

Owing to spin restrictions in the process of recombination of the electrons and holes in the emitter layer of organic electroluminescent devices, fluorescent emitters can convert only a maximum of 25% of the electrical energy to light. The use of organometallic complex structures draws benefit from the triplet excitons that are otherwise lost to electroluminescence via radiationless quenching processes. Phosphorescent emitters that contain particularly iridium or other transition metals are state of the art. These phosphorescent emitters are doped into matrix materials with high-lying triplet states, these matrix materials frequently comprising carbazole derivatives, for example bis(carbazolyl)biphenyl, and also ketones (WO 2004/093207), phosphine oxides, sulfones (WO 2005/003253), or triazine compounds such as triazinylspirobifluorene (WO 2005/053055 and WO 2010/05306).

In materials in which the triplet state is at high energy and hence close to the lowest excited singlet state, however, there can be reverse intersystem crossing (RISC). This raises triplet excitons thermally into the singlet state, where they can contribute to fluorescence. This is referred to as thermally activated delayed fluorescence (TADF). In this way, it is theoretically possible for up to 100% of the energy stored in the energetically excited states to be emitted in the form of fluorescent electroluminescence.

Organic molecules composed of donor and acceptor structures where the highest occupied molecular orbital (HOMO) and lowest unoccupied molecular orbital (LUMO) have only slight overlap have small splittings between the lowest excited singlet and the lowest triplet, and so the TADF process can proceed efficiently. Therefore, molecules of this kind are an efficient and inexpensive alternative to organometallic phosphorescent emitter materials.

In the context of the present invention, it has been found that the compounds as described herein are of excellent suitability for use in optoelectronic components, especially as emitter materials or matrix materials. Some of the compounds described herein are notable for a small energy splitting between the lowest excited triplet and the lowest excited singlet, and for that reason it is possible in such materials, via the mechanism of thermally activated delayed fluorescence (TADF), to exploit a reverse transition from the triplet system to the singlet system in order to convert some of the triplet states to singlets by thermal excitation. These singlet states then in turn contribute to radiative recombination, which correspondingly increases the internal quantum efficiency of an OLED above 25%. In addition, materials where both the lowest excited singlet and the lowest excited triplet are at very high energies are proposed, but where the splitting between these two excitations is also relatively large. Owing to the large splitting between singlet and triplet, these materials are unsuitable as TADF emitters, but they can be utilized as transport materials and matrix materials for TADF emitters. Particularly the high value for the lowest triplet excitation in such a matrix material prevents a triplet state of an embedded TADF emitter or of an embedded phosphorescent emitter from being converted to a non-radiative triplet state of the matrix material at higher energy.

In a first aspect, the present invention is therefore directed to a compound comprising at least one donor group and at least one acceptor group, in which the vertical transition energy of the lowest excited triplet back to the electronic ground state both for the individual corresponding donor molecule and for the individual corresponding acceptor molecule is at least 2.2 eV.

In a further aspect, the present invention is directed to the use of a compound as described herein in an optoelectronic component, for example an organic electroluminescent device (OLED), an organic integrated circuit (O-IC), an organic field-effect transistor (O-FET), an organic thin-film transistor (O-TFT), an organic light-emitting transistor (O-LET), an organic solar cell (O-SC), an organic optical detector, an organic photoreceptor, an organic field-quench device (O-FOD), a light-emitting electrochemical cell (LEC) or an organic laser diode (O-Laser) as emitter or matrix material.

Finally, the present invention is also directed to an optoelectronic component comprising at least one compound as described herein.

“At least one”, as used herein, means 1 or more, i.e. 1, 2, 3, 4, 5, 6, 7, 8, 9 or more. “At least one donor group” thus means, for example, at least one kind of donor group, i.e. what may be meant may be one kind of donor group or a mixture of multiple different donor groups.

The present invention relates to metal-free compounds that can be used both as emitter materials and as matrix materials in optoelectronic and electronic components. A compound as described herein comprises at least one donor group and at least one acceptor group in which the transition energy of the lowest excited triplet back to the electronic ground state both for the corresponding donor molecule and for the corresponding acceptor molecule is at least 2.2 eV.

This opens up the possibility that the recombination of the internal charge transfer state between donor group and acceptor group back to the electronic ground state is also still at a very high transition energy.

The expression “transition energy of the lowest excited triplet” refers here to the energy corresponding to the vertical transition energy of the transition from the lowest relaxed triplet configuration T₁ back to the electronic ground state S₀ in a molecule. Such a vertical transition energy Eᵥₑᵣₜ(T₁→S₀) from the minimum of the excited potential surface in the electron configuration T₁ can be determined from the phosphorescence spectrum I(E) in proportion to I(λ)/E². This phosphorescence spectrum I(E) is determined by the possible final states in the molecule and the density of states of the emitted photons, and so a distinction has to be made between the average energy of the measured intensity I(E) on the one hand and the average energy of the scaled intensity I(E)/E³ on the other hand. The energy average of the scaled intensity I(E)/E³ corresponds specifically to the vertical transition energy from the minimum of the potential surface T₁ of the emitter back to the potential surface S₀. Therefore, this operative definition of vertical transition energy Eᵥₑᵣₜ(T₁→S₀) from a measured phosphorescence spectrum allows a direct comparison with a T₁→S₀ transition energy calculated by theoretical methods from the minimum of the potential surface T₁ of the emitter back to the electronic ground state S₀, calculated, for example, by time-dependent density functional theory. By contrast, the transition from the minimum of the electronic ground state S₀ to the lowest triplet state T₁, by virtue of Its vanishingly small oscillator intensity, is not visible in absorption, and so it is not possible to experimentally verify a value for the S₀→T₁ transition energy calculated by time-dependent density functional theory.

In accordance with the above definition, it is easily possible to ascertain the T₁→S₀ transition energy from a phosphorescence spectrum and, correspondingly, the S₁→S₀ transition energy from a fluorescence spectrum, irrespective of the line shape, broadening and visibility of vibronic sub-bands. This transition energy directly gives the correct energy gap between the minimum of the T₁ potential surface and the S₀ potential surface of the molecule in the same T₁ geometry, or, correspondingly, the correct energy gap between the minimum of the S₁ potential surface and the S₀ potential surface in the same S₁ geometry. This definition of the transition energy is thus clearly superior to other possible methods of ascertaining the energy of the T₁→S₀ triplet transition or of the S₁→S₀ singlet transition, for example:
the ascertaining of the vibronic sub-band E₀₀ at the shortest wavelength or highest energy, because the visibility of the sub-bands can be restricted by different degrees of broadening; cf. Endo et al, Appl. Phys. Lett. 98, 083302 (2012)
calculations of the gap between the potential surfaces in the geometry of the S₀ ground state; cf. Lee et at., Appl. Phys. Lett. 101, 093306 (2012)
tangent construction on the energy dependent emission spectrum I(E), in order to interpolate the minimum of the spectrum at high energy; cf. Hirata et al., Nature Materials 124, 330 (2015)
tangent construction on the wavelength-dependent emission spectrum I(λ), in order to interpolate the minimum of the spectrum at low wavelength (i.e., high energy); cf. Tanaka et al, Jpn. J. Appl. Phys. 46, L117 (2007).

In molecules formed from suitably selected donor groups and acceptor groups, owing to a small splitting between the lowest excited triplet and the lowest excited singlet, it is possible as described above to exploit a reverse transition from the triplet system to the singlet system via the mechanism of thermally activated delayed fluorescence in order to convert some of the triplet states to singlets by thermal excitation. These singlet states then in turn contribute to radiative recombination, which correspondingly increases the internal quantum efficiency of OLEDs over and above the otherwise customary 25%.

In one embodiment, the present invention is additionally directed to compounds in which the dihedral angle between the at least one donor group and the at least one acceptor group in the electronic ground state is at least 70°.

In the lowest charge transfer state between donor group and acceptor group, the splitting between singlet and triplet depends to a crucial degree on this dihedral angle between the two groups, such that a sufficiently large dihedral angle brings about a small splitting. Suitable methods of determining this dihedral angle are firstly the determination of the molecular geometry in a crystalline phase by x-ray diffraction or secondly the calculation of the geometry of the free molecule by sufficiently exact quantum-chemical methods, for example density-functional theory.

For the nonplanar donor groups and acceptor groups of some of the compounds described herein, the reference plane is defined as follows. As described hereinafter, the ligands are each bonded to a carbon atom of another part of the molecule via a nitrogen atom or phosphorus atom. This carbon atom is part of an aromatic group of the other part of the molecule. For the definition of the dihedral angle about the carbon-nitrogen (or carbon-phosphorus) bond to the ligand, the obvious plane of the aromatic group of the part of the molecule that contains the carbon atom of the carbon-nitrogen (or carbon-phosphorus) bond is employed, meaning that planar parts of the molecule at a relatively high distance from this bond that are possibly differently oriented are not taken into account. The nitrogen atom or phosphorus atom of the ligand, by contrast, is in a nonaromatic and nonplanar hexagon which is continued by two phenyl groups, in each of which one carbon-carbon bond is shared with the central hexagon. The non-coplanar planes of the two aromatic phenyl groups can be used to define two angle-halving planes. The reference plane for the ligand is defined as the angle-halving plane that forms a relatively small angle with the aromatic groups. For the example of phenothiazine dioxide, the angle between the aromatic phenyl planes and the reference plane as defined above is about 13°.

In various embodiments of the invention, the compounds of the invention show thermally activated delayed fluorescence and are therefore suitable as TADF emitters.

In one embodiment, the present invention additionally relates to a compound in which at least one donor group and/or acceptor group is a compound of the formula (I) or a compound of the formula (II):

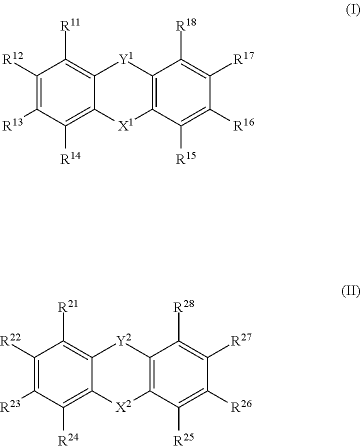

In the compounds of the formula (I), R¹¹-R¹⁸ are each independently selected from the group consisting of hydrogen, C₁-C₂₀ alkyl, aryl and bromine, or R¹¹, R¹⁴, R¹⁵ and R¹⁸ are as defined above and R¹³ and/or R¹⁶ or R¹² and/or R¹⁷ denote(s)

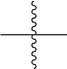

which represents the point of attachment to another part of the molecule. Y¹ is selected from the group consisting of N—R¹⁹, P(O)R¹⁹ and P(O)OR¹⁹. X¹ is selected from the group consisting of P(O)R¹⁹, P(O)OR¹⁹, Si(C₁-C₂₀ alkyl)₂, Si(aryl)₂ and SO₂, where R¹⁹ is in each case independently selected from the group consisting of hydrogen, C₁-C₂₀ alkyl and aryl, or R¹⁹ denotes

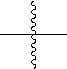

which represents the point of attachment to another part of the molecule.

In the compounds of the formula (II), R²¹-R²⁸ are each independently selected from the group consisting of hydrogen, C₁-C₂₀ alkyl, aryl and bromine, or R²¹, R²⁴, R²⁵ and R²⁸ are as defined above and R²³ and/or R²⁶ or R²² and/or R²⁷ denote(s)

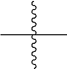

which represents the point of attachment to another part of the molecule. Y² is selected from the group consisting of P(O)R²⁹, P(O)OR²⁹, Si(C₁-C₂₀ alkyl)₂, Si(aryl)₂ and SO₂. X² is selected from the group consisting of P(O)R²⁹, P(O)OR²⁹, Si(C₁-C₂₀ alkyl)₂, Si(aryl)₂ and SO₂, where R²⁹ is in each case independently selected from the group consisting of hydrogen, C₁-C₂₀ alkyl and aryl, or R²⁹ denotes

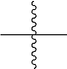

which represents the point of attachment to another part of the molecule.

A “C₁-C₂₀ alkyl”, as used herein, denotes a linear or branched hydrocarbyl group having 1 to 20 carbon atoms. Particular examples include, without restriction, groups that are selected from the group consisting of methyl, ethyl, propyl, isopropyl, c-propyl, n-butyl, sec butyl, isobutyl and t-butyl groups, where the aforementioned groups may each be substituted or unsubstituted. When they are substituted, the substituents are preferably selected from the group consisting of halogens and pseudohalogens (—ON, —N₃, —OCN, —NCO, —CNO, —SCN, —NCS, —SeCN). In various embodiments, the alkyl groups are unsubstituted. In various embodiments, the alkyl groups are C1-4 alkyl groups.

An “aryl”, as used herein, denotes either a monocyclic aromatic group, for example phenyl or a fused (annelated, polycyclic) aromatic polycyclic group, for example naphthalenyl or phenanthrenyl. A fused (annelated, polycyclic) aromatic polycycle in the context of the present application consists of two or more simple (monocyclic) aromatic rings fused to one another. Particular examples include, without restriction, groups that are selected from the group consisting of phenyl, naphthyl, anthracenyl, phenanthrenyl, fluorenyl, pyrenyl, dihydropyrenyl, chrysenyl, perylenyl, fluoranthenyl, benzanthracenyl, benzphenanthrenyl, tetracenyl, pentacenyl and benzpyrenyl, where the aforementioned groups may each be substituted or unsubstituted. When they are substituted, the substituents are preferably selected from the group consisting of halogens and pseudohalogens (—CN, —N₃, —OCN, —NCO, —CNO, —SCN, —NCS, —SeCN). In various embodiments, the aryl groups are unsubstituted. Particular preference is given to phenyl.

In one embodiment, in the compound, as described herein, R¹⁹ and/or R²⁹ is/are each independently selected from the group consisting of hydrogen, methyl and phenyl, or R¹⁹ and/or R²⁹ denote(s)

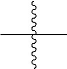

which represents the point of attachment to another part of the molecule.

In various embodiments, at least one donor group and/or acceptor group is a compound selected from the group consisting of dihydroacridine, dimethylacridine, phenothiazine or phenoxazine, where at least one further part of the molecule is chosen in accordance with the formulae (I) or (II).

In various embodiments, the compound, as described herein, in addition to the at least one donor group and the at least one acceptor group, comprises at least one further donor group or acceptor group in which the energy of the lowest excited triplet is at least 2.2 eV, as defined above.

In various embodiments, the at least one further donor group or acceptor group of the compound described herein is a compound of the formula (I) or (II), as defined above.

In various embodiments, the compound is a compound of the formula (III) or a compound of the formula (IV):

A-B-A (III)

A-B (IV)

In the compounds of the formula (HI) or (IV), components A and B are each either a donor group or an acceptor group, where at least one A or Bis, preferably all A and more preferably all A and B are each, independently a compound having a vertical transition energy of the lowest excited triplet back to the electronic ground state of at least 2.2 eV, as defined above. It is possible here, for example, for all A to be a donor group and B to be an acceptor group, or vice versa. In various embodiments, the dihedral angle between at least one donor group and at least one acceptor group, i.e. preferably between (each) A and B, is at least 70⁰. A and B are each bonded to one another via covalent bonds, where respective points of attachment may be those that have been described above in the context of the formulae (I) and (II). A is preferably bonded via the group corresponding to R¹⁹ in formula (I), and B preferably via the group corresponding to R¹³ and/or R¹⁶ or R¹² and/or R¹⁷ in formula (I) or R²³ and/or R²⁶ or R²² and/or R²⁷ in formula (II).

In various embodiments, (i) component A is in each case independently a compound of the formula (I), as defined above, which is bonded to component B via R¹⁹ in each case. In such embodiments, component B may be a compound having a vertical transition energy of the lowest excited triplet back to the electronic ground state of at least 2.2 eV, as defined above, especially selected from carbazole and carbazole-containing compounds, dihydroacridine, dimethylacridine, phenothiazine and phenoxazine, even more preferably from dihydroacridine, dimethylacridine, phenothiazine and phenoxazine. It will be appreciated that A in each case is the corresponding radicals of the aforementioned compounds, i.e. dihydroacridinyl etc. For the sake of simplicity, however, the name of the compound as such is also used as an alternative, it being clear to the person skilled in the art that what is meant in each case is the corresponding radical. When B is carbazole or a carbazole-containing compound, it is preferable that the carbazole has a sterically demanding side group, especially one that ensures that the dihedral angle between each A and B is at least 70° . In various embodiments, the dihedral angle between at least one donor group and at least one acceptor group, i.e. preferably between (each) A and B, is at least 70° . As already mentioned above, B in these embodiments is preferably bonded via the group in the aforementioned radicals that corresponds to R¹³ and/or R¹⁶ or R¹² and/or R¹⁷ in formula (I).

In various embodiments, component Bis a compound of the formula (I), as defined above, or a compound of the formula (a), as defined above, each of which is bonded to components A via (a) R¹³ and/or R¹⁶ or (b) R¹² and/or R¹⁷ or (c) R²³ and/or R²⁶ or (d) R²² and/or R²⁷. It is possible here for each component A to be independently selected from compounds having a vertical transition energy of the lowest excited triplet back to the electronic ground state of at least 2.2 eV, as defined above, especially selected from carbazole and carbazole-containing compounds, dihydroacridine, dimethylacridine, phenothiazine and phenoxazine, even more preferably from dihydroacridine, dimethylacridine, phenothiazine and phenoxazine. When A is carbazole or a carbazole-containing compound, it is preferable that the carbazole has a sterically demanding side group, especially one that ensures that the dihedral angle between each A and B is at least 70° . In preferred embodiments, A is a compound of the formula (I), as defined above, bonded in each case to component B via R¹⁹. In various embodiments, the dihedral angle between at least one donor group and at least one acceptor group, i.e. preferably between (each) A and B, is at least 70⁰. As already mentioned above, A in these embodiments is preferably bonded via the group of the aforementioned radicals that corresponds to R¹⁹ in formula (I).

In various embodiments of the invention, in the compounds of the formulae (III) and (IV), (1) all A are the same and B are different from A or (2) all A and B are the same. In various embodiments, all A and/or B are selected from compounds of the formula (I) as defined above. In this case, Y¹ is preferably NR⁹ and X¹ is Si(C₁-C₂₀ alkyl)₂ or SO₂, where alkyl is preferably methyl and/or R¹⁹ is preferably H, methyl or ethyl. In particularly preferred embodiments, B is a compound of the formula (I), where Y¹ is preferably NR¹⁹ and X¹ is Si(C₁-C₂₀ alkyl)₂ or SO₂, where alkyl is preferably methyl and/or R¹⁹ is preferably H, methyl or ethyl, and A or both A, which are preferably the same, are dihydroacridine, dimethylacridine, phenothiazine or phenoxazine. In the aforementioned embodiments, B is preferably bonded to the nitrogen atom of A via (a) R¹³ and/or R¹⁶ or (b) R¹² and/or R¹⁷.

In various embodiments, the compound is selected from the group of compounds of the formulae (1)-(14):

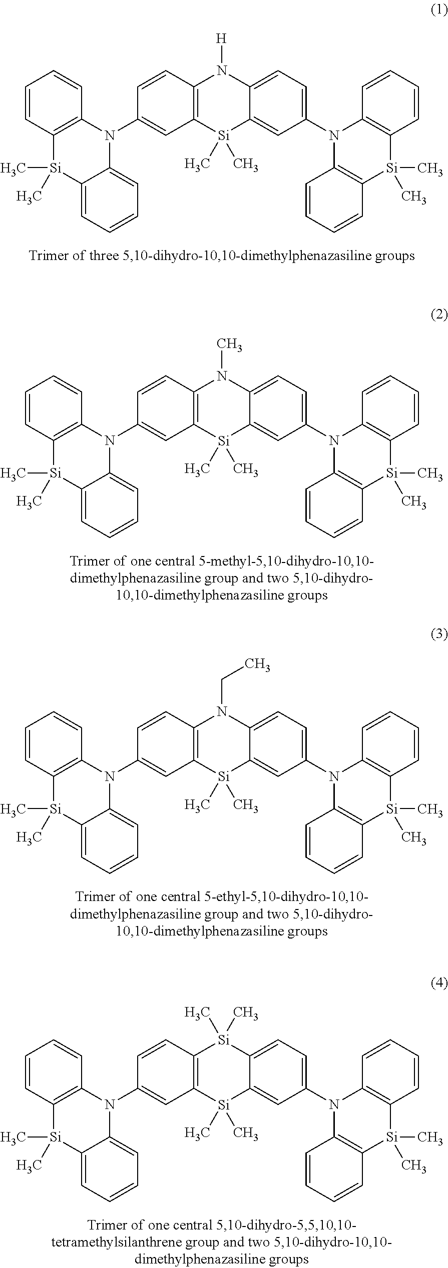

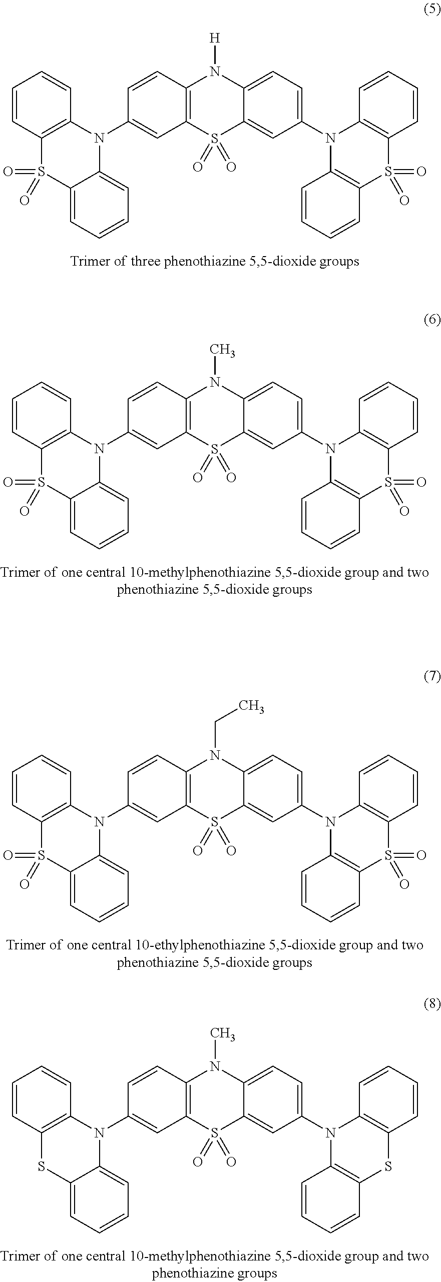 [SMILES: O=S1(=O)c2cc(N3c4ccccc4S(=O)(=O)c4ccccc43)ccc2Nc2ccc(N3c4ccccc4S(=O)(=O)c4ccccc43)cc21 ; CN1c2ccc(N3c4ccccc4S(=O)(=O)c4ccccc43)cc2S(=O)(=O)c2cc(N3c4ccccc4S(=O)(=O)c4ccccc43)ccc21 ; CCN1c2ccc(N3c4ccccc4S(=O)(=O)c4ccccc43)cc2S(=O)(=O)c2cc(N3c4ccccc4S(=O)(=O)c4ccccc43)ccc21 ; CN1c2ccc(N3c4ccccc4Sc4ccccc43)cc2S(=O)(=O)c2cc(N3c4ccccc4Sc4ccccc43)ccc21]

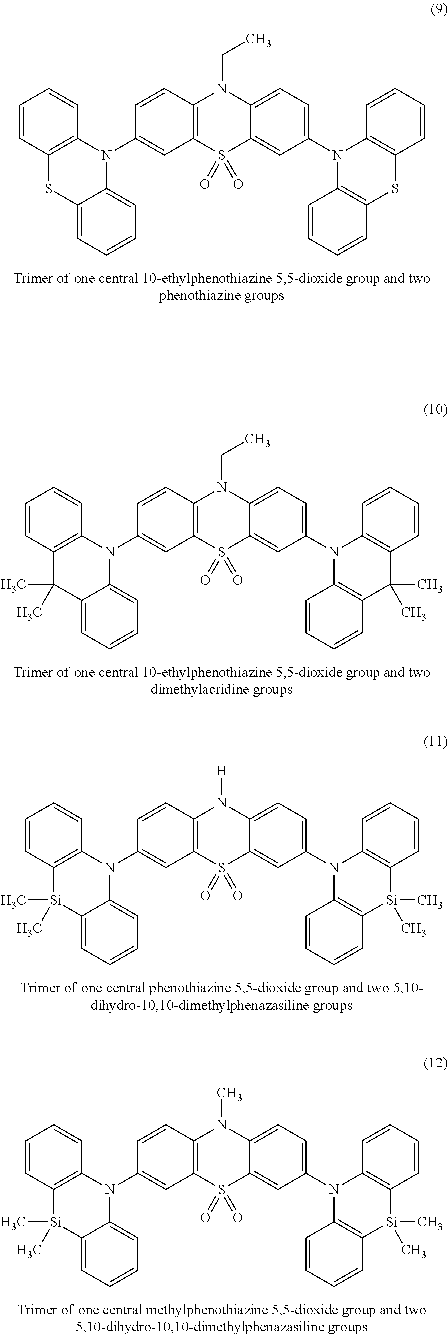

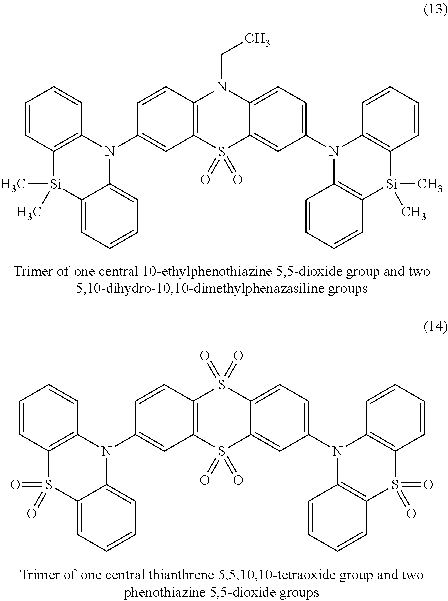

In various embodiments of the invention, the compound of the formula A-B or A-B-A, i.e. of the formula (III) or (IV), is free of carbazole groups or of carbazole-derived groups having a carbazole base structure, i.e. especially substituted carbazoles and in this context especially those having substituents that are not very sterically demanding.

In some embodiments of the invention, the compounds of the invention are not:

(1) a trimer of the formula (III) composed of a central phenothiazine 5,5-dioxide as B, and two 9,9-dimethyl-9,10-dihydroacridine (DMAC) groups as A, where the groups are each unsubstituted;

(2) a dimer of the formula (IV) composed of phenothiazine 5,5-dioxide as B and a 9,9-dimethyl-9,10-dihydroacridine (DMAC) group as A, where the groups are each unsubstituted;

(3) a trimer of the formula (III) composed of a central phenothiazine 5,5-dioxide as B, and two carbazole groups as A, where the groups are each unsubstituted;

(4) a dimer of the formula (IV) composed of phenothiazine 5,5-dioxide as B and a carbazole group as A, where the groups are each unsubstituted;

(5) 10,10′-bis(4-tert-butylphenyl)-10H,10′H(3,3′)-biphenothiazine (t-BPBP);

(6) 10-(4-tert-butylphenyl)-10H-phenothiazine;

(7) 3-bromo-10-(4-tert-butylphenyl)-10H-phenothiazine;

(8) 10-(4-tert-butylphenyl)-3-((4,4,5,5-tetramethyl)-[1.2.3]dioxyborolan-2-yl)-10-phenothiazine;

(9) 10′-(4-tert-butylphenyl)-10′H[10,3′;7′10″]-terphenothiazine (t-BPTP);

(10) 3,7-dibromo-10-(4-tert-butylphenyl)-10H-phenothiazine;

(11) 7,7′-diphenothiazyl-10,10′-bis(4-tert-butylphenyl)-10H,10′H[3,3]-biphenothiazine (DP-t-BPBP);

(12) 7,7′-dibromo-10,10′-bis(4-tert-butylphenyl)-10H,10′H[3,3π-biphenothiazine;

(13) 1,4-diphenothiazylbenzene; or

(14) 2,9,10-diphenothiazylanthracene.

The present invention further relates to the use of a compound as described herein in an optoelectronic component, for example an organic electroluminescent device (OLED), an organic integrated circuit (O-IC), an organic field-effect transistor (O-FET), an organic thin-film transistor (O-TFT), an organic light-emitting transistor (O-LET), an organic solar cell (O-SC), an organic optical detector, an organic photoreceptor, an organic field-quench device (O-FOD), a light-emitting electrochemical cell (LEC) or an organic laser diode (O-laser) as emitter or matrix material.

Finally, the present invention also provides an optoelectronic component comprising at least one compound as described herein.

In various embodiments, the optoelectronic component comprises OSCs having a photoactive organic layer. This photoactive layer includes low molecular weight compounds, oligomers, polymers or mixtures thereof as organic coating materials. A preferably opaque or semitransparent electrode has been applied as outer contact layer to this thin-layer component.

In a further embodiment of the invention, the optoelectronic component is disposed on a flexible substrate.

In the context of the present invention, a flexible substrate is understood to mean a substrate which assures deformability as a result of external forces. This makes flexible substrates of this kind suitable for arrangement on curved surfaces as well. Flexible substrates include, for example, plastic films or metal foils, but are not limited thereto.

In various embodiments, the coating for production of an optoelectronic component is effected by means of vacuum processing of the organic compounds of the invention. In various embodiments, the compounds of the invention used for production of the optoelectronic component therefore have a low evaporation temperature, preferably <300° C., more preferably <250° C., but not lower than 150° C. In various embodiments, however, the evaporation temperature is at least 120° C. It is particularly advantageous when the organic compounds of the invention are sublimable under high vacuum.

In a further configuration of the present invention, it may be the case that the coating for production of an optoelectronic component is effected by means of solution processing of the compounds described herein. The availability of commercial spray robots means that this application method can advantageously be scaled up in a simple manner to the industrial scale in roll-to-roll methods.

In various embodiments, the optoelectronic component, in the context of the present invention, is a solar cell of the generic type. Such an optoelectronic component typically has a layer structure wherein the respective lowermost and uppermost layers are configured as electrode and counterelectrode for formation of electrical contacts. In various embodiments, the optoelectronic component is arranged on a substrate, for example glass, plastic (PET, etc.) or a metal ribbon. At least one organic layer comprising at least one organic compound is arranged between the near-substrate electrode and the counterelectrode. Organic compounds used here may be organic low molecular weight compounds, oligomers, polymers or mixtures. In various embodiments, the organic layer is a photoactive layer. In various embodiments of the photoactive layer, it may be designed, for example, in the form of a mixed layer composed of an electron donor material and an electron acceptor material. Charge carrier transport layers may be arranged adjacent to the at least one photoactive layer. According to the configuration, these can preferably transport electrons (=negative charges) or holes (=positive charges) from or to the respective electrodes. In various embodiments, the optoelectronic component is configured as a tandem or multiple component. In this case, at least two optoelectronic components are deposited one on top of another as a layer system. In various embodiments, it is possible for there to be adjoining additional layers for coating or encapsulation of the component or further components on or beneath the base layers and outer layers configured as contacts.

In one embodiment of the invention, the organic layer takes the form of one or more thin layers of vacuum-processed low molecular weight compounds or organic polymers. The prior art discloses a multitude of optoelectronic components based on vacuum-processed low molecular weight compounds and polymers (Walzer et al., Chemical Reviews 2007, 107(4), 1233-1271; Peumans et al., J. Appl. Phys. 2003, 93(7), 3693-3722).

Preferably, the organic layer is deposited on a substrate using vacuum-processible compounds of the compounds of the invention described herein. In an alternatively preferred embodiment of the present invention, the organic layer is deposited on a substrate by wet-chemical means using solutions.

Typical examples of optoelectronic components comprising compounds of the invention as described above are likewise provided. In embodiments of this kind, the compound of the invention, in various embodiments, is selected from the group consisting of compounds (1)-(14) as defined above.

In various other embodiments of the present invention, the optoelectronic component comprising at least one of the compounds as described herein is an organic light-emitting diode (OLED).

The OLEDs of the invention are n principle formed from several layers, for example:

1. anode

2. hole conductor layer

3. blocking layer for electrons/excitons

4. light-emitting layer

5. blocking layer for holes/excitons

6. electron conductor layer

7. cathode

Layer sequences other than the aforementioned structure are also possible, these being known to those skilled in the art. For example, it is possible that the OLED does not have all the layers mentioned; for example, an OLED having the layers (1) (anode), (4) (light-emitting layer) and (7) (cathode) is likewise suitable, in which case the functions of layers (2) (hole conductor layer), (3) (blocking layer for electrons/excitons), (5) (blocking layer for holes/excitons) and (6) (electron conductor layer) are assumed by the adjoining layers. OLEDs having layers (1), (2), (4) and (7) or layers (1), (4), (5), (6) and (7) are likewise suitable. In addition, the OLEDs may have a blocking layer for electrons/excitons between the anode (1) and the hole conductor layer (2).

The compounds, as described herein, may find use as emitter materials or matrix materials in the light-emitting layer.

The compounds, as described herein, may be present as the sole emitter material and/or matrix material—without further additions—in the light-emitting layer. However, it is likewise possible that, as well as the compounds, as described herein, that are used in accordance with the invention, further compounds are present in the light-emitting layer. For example, one or more fluorescent dyes may be present in order to alter the emission color of the emitter molecule present. In addition, it is possible to use a diluent material. This diluent material may be a polymer, for example poly(N-vinylcarbazole) or polysilane. However, the diluent material may likewise be a small molecule, for example 4,4′-N,N′-dicarbazolebiphenyl (CBP CDP) or tertiary aromatic amines.

The individual layers of the OLED among those mentioned above may in turn be formed from 2 or more layers. For example, the hole-transporting layer may be formed from a layer into which holes are injected from the electrode, and a layer which transports the holes away from the hole-injecting layer into the light-emitting layer. The electron-transporting layer may likewise consist of multiple layers, for example a layer in which electrons are injected by the electrode, and a layer which receives electrons from the electron-injecting layer and transports them into the light-emitting layer. These said layers are each selected according to factors such as energy level, thermal resistance and charge carrier mobility, and energy differential of the layers mentioned with the organic layers or the metal electrodes. The person skilled in the art will be able to choose the construction of the OLEDs such that it is optimized for the organic compounds used in accordance with the invention as emitter substances.

In order to obtain particularly efficient OLEDs, the HOMO (highest occupied molecular orbital) of the hole-transporting layer should be matched to the work function of the anode and the LUMO (lowest unoccupied molecular orbital) of the electron-transporting layer should be matched to the work function of the cathode.

The anode (1) is an electrode which provides positive charge carriers, It may be formed, for example, from materials including a metal, a mixture of various metals, a metal alloy, a metal oxide or a mixture of various metal oxides. Alternatively, the anode may be a conductive polymer. Suitable metals include the metals of groups 11, 4 and 5 of the Periodic Table of the Elements and the transition metals of groups 9 and 10. If the anode is to be transparent, in general, mixed metal oxides of groups 12, 13 and 14 of the Periodic Table of the Elements are used, for example indium tin oxide (ITO). It is likewise possible that the anode (1) comprises an organic material, for example polyaniline, as described, for example, in Nature, vol. 357, pages 477 to 479 (11 June 1992). At least either the cathode or the anode should be at least partly transparent in order to be able to outcouple the light emitted. Preferably, the material used for the anode (1) is ITO,

Suitable hole conductor materials for layer (2) of the OLEDs of the invention are disclosed, for example, in Kirk-Othmer Encyclopedia of Chemical Technology, 4th edition, vol. 18, pages 837 to 860, 1996. Both hole-transporting molecules and polymers can be used as hole transport material. Customarily used hole-transporting molecules are selected, for example, from the group consisting of tris-[N-(1-naphthyl)-N-(phenylamino)]triphenylamine (1-NaphDATA), 4,4′-bis[N-(1-naphthyl)-N-phenyl-amino]biphenyl (α-NPD), N,N′-diphenyl-N,N′-bis(3-methylphenyl)-[1,1′-biphenyl]-4,4′-diamine (TPD), 1,1-bis[(di-4-tolylamino)phenyl]cyclohexane (TAMC), N,N′-bis(4-methylphenyl)-N,N′-bis(4-ethylphenyl)-[1,1′-(3,3′-dimethyl)biphenyl]-4,4′-diamine (ETPD), tetrakis-(3-methylphenyl)-N,N,N′,N′-2,5-phenylendiamine (PDA), α-phenyl-4-N,N-diphenylaminostyrene (TPS), p-(diethylamino)-benzaldehyde diphenylhydrazone (DEH), triphenylamine (TPA), bis[4-(N,N-diethylamino)-2-methylphenyl)(4-methyl-phenyl)methane (MPMP), 1-phenyl-3-[p-(diethylamino)styryl]-5-[p-(diethylamino)phenyl]pyrazoline (PPR or DEASP), 1,2-trans-bis(9H-carbazol-9-yl)cyclobutane (DCZB), N,N,N′,N′-tetrakis(4-methylphenyl)-(1,1′-biphenyl)-4,4′-diamine (TIB), 4,4′,4″-tris(N,N-diphenylamino)triphenylamine (TDTA), porphyrin compounds and phthalocyanines such as copper phthalocyanines. Customarily used hole-transporting polymers are selected, for example, from the group consisting of polyvinylcarbazoles, (phenylmethyl)polysilanes and polyanilines. It is likewise possible to obtain hole-transporting polymers by doping hole-transporting molecules into polymers such as polystyrene and polycarbonate. Suitable hole-transporting molecules are the molecules already mentioned above.

In addition, it is possible in various embodiments to use carbene complexes as hole conductor materials, where the band gap of the at least one hole conductor material is generally greater than the band gap of the emitter material used. In the context of the present application, “band gap” is understood to mean the triplet energy. Suitable carbene complexes are, for example, carbene complexes as described in WO 2005/019373 A2, WO 2006/056418 A2 and WO 2005/113704, and in the prior European applications EP 06112228.9 and EP 06112198.4 that were yet to be published at the priority date of the present application.

The light-emitting layer (4) comprises at least one emitter material. The emitter may in principle be a fluorescence or phosphorescence emitter, suitable emitter materials being known to those skilled in the art. Preferably, the at least one emitter material is a material that enables thermally activated delayed fluorescence. At least one of the emitter materials present in the light-emitting layer (4) here is a compound as described herein. Furthermore, it is possible to use at least one compound as described herein additionally as matrix material. Alternatively, commonly used matrix materials that are customary in the prior art are known to those skilled in the art. Illustrative matrix materials are selected from the classes of the oligoarylenes (e.g. 2,2′,7,7′-tetraphenylspirobifluorene or dinaphthylanthracene), especially the oligoarylenes containing fused aromatic groups, for example anthracene, benzanthracene, benzphenanthrene, phenanthrene, tetracene, coronene, chrysene, fluorene, spirofluorene, perylene, phthaloperylene, naphthaloperyiene, decacyclene, rubrene, the oligoarylenevinylenes (e.g. DPVBi=4,4′-bis(2,2-diphenylethenyl)-1,r-biphenyl or spina-DPVBi according to EP 676461), or the polypodal metal complexes, especially metal complexes of 8-hydroxyquinoline, e.g. Alq_{:3} (=aluminum(III) tris(8-hydroxyquinoline)) or bis(2-methyl-8-quinolinolato)-4-(phenylphenolinolato)aluminum. In general, suitable matrix materials are known to the person skilled in the art, for example, from Organic. Light-Emitting Materials and Devices (Optical Science and Engineering Series; Ed.: Z. Li, H. Meng, CRC Press Inc., published 2006).

The blocking layer for holes/excitons (5) may include hole blacker materials typically used in OLEDs, such as 2,9-dimethyl-4,7-diphenyl-1,10-phenanthroline (bathocuproin, (BCP)), bis-(2-methyl-8-quinolinato)-4-phenylphenylato)aluminum(III) (BAIq), phenothiazine S,S-dioxide derivatives and 1,3,5-tris(N-phenyl-2-benzylimidazol)benzene (TPBI), and TPBI and BAIq are also suitable as electron-conducting materials. In a further embodiment, it is possible to use compounds containing aromatic or heteroaromatic rings bonded via groups containing carbonyl groups, as disclosed in WO2006/100298, as blocking layer for holes/excitons (5) or as matrix materials in the light-emitting layer (4).

In various embodiments, the present invention relates to an OLED of the invention comprising the following layers: (1) anode, (2) hole conductor layer, (3) blocking layer for electrons/excitors, (4) light-emitting layer, (5) blocking layer for holes/excitons, (6) electron conductor layer and (7) cathode, and optionally further layers, where the light-emitting layer (4) comprises at least one compound of the formula (I).

Suitable electron conductor materials for the layer (6) of the OLEDs of the invention include metals chelated to oxinoid compounds, such as tris(8-quinolinolato)aluminum (Alq₃), bis-(2-methyl-8-quinolinato)-4-(phenylphohylato)aluminum(III) (BAIq), phenanthroline-based compounds such as 2,9-dimethyl-4,7-diphenyl-1,10-phenanthroline (DDPA=BCP) or 4,7-diphenyl-1,10-phenanthroline (DPA) and azole compounds such as 2-(4-biphenylyl)-5-(4-t-butylpnenyl)-1,3,4-oxadiazole (PBD) and 3-(4-biphenylyl)-4-phenyl-5-(4-t-butylpnenyl)-1,2,4-triazole (TAZ) and 2,2″,2″-(13,5-phenylene)tris-[1-phenyl-1H-benzimidazole] (TPBI). It is possible here for the layer (5) to serve both to facilitate electron transport and as a buffer layer or as a barrier layer in order to prevent quenching of the exciton at the interfaces of the layers of the OLED. Preferably, the layer (5) improves the mobility of the electrons and reduces quenching of the exciton. Electron conductor materials suitable with preference are TPBI and BAIq.

Some of the materials mentioned above as hole conductor materials and electron conductor materials can fulfill multiple functions. For example, some of the electron-conducting materials are simultaneously hole-blocking materials if they have a low-lying HOMO. These may be used, for example, in the blocking layer for holes/excitons (5). However, it is likewise possible that the function as hole/excitons blacker is assumed by the layer (6), such that the layer (5) can be dispensed with.

The charge transport layers may also have been electronically doped in order to improve the transport properties of the materials used, in order firstly to make the layer thicknesses more generous (avoidance of pinholes/short-circuits) and secondly to minimize the operating voltage of the component. For example, the hole conductor materials can be doped with electron acceptors; for example, phthalocyanines or arylamines such as TPD or TDTA can be doped with tetrafluoro-tetracyanoquinodimethane (F4-TCNQ). The electron conductor materials can be doped, for example, with alkali metals, for example Alq₃ with lithium. Electronic doping is known to those skilled in the art and is disclosed, for example, in W. Cao, A. Kahn, J. Appl. Phys., vol. 94, no. 1, 1 July 2003 (p-doped organic layers); A. G. Werner, F. Li, K. Harada, M. Pfeiffer, T. Fritz, K. Leo. Appl, Phys. Lett., vol. 82, no. 25, 23 Jun. 2003 and Pfeiffer et al., Organic Electronics 2003, 4, 89-103.

The cathode (7) is an electrode that serves to introduce electrons or negative charge carriers. Suitable materials for the cathode are selected from the group consisting of alkali metals of group la, for example Li, Cs, alkaline earth metals of group Ha, for example calcium, barium or magnesium, metals of group IIb of the Periodic Table of the Elements (old ILJPAC version), comprising the lanthanides and actinides, for example samarium. In addition, it is also possible to use metals such as aluminum or indium, and combinations of all the metals mentioned. In addition, lithium-containing organometallic compounds or LiF may be applied between the organic layer and the cathode in order to reduce the operating voltage.

The OLED according to the present invention may additionally comprise further layers that are known to those skilled in the art. For example, a layer that facilitates the transport of the positive charge and/or adjusts the band gap of the layers with respect to one another may be applied between the layer (2) and the light-emitting layer (4). Alternatively, this further layer may serve as a protective layer, In an analogous manner, it is possible for additional layers to be present between the light-emitting layer (4) and the layer (5), in order to facilitate the transport of the negative charge and/or to adjust the band gap between the layers relative to one another. Alternatively, this layer can serve as protective layer.

In various embodiments, the OLED of the invention, in addition to layers (1) to (7), comprises at least one of the following further layers:

a hole injection layer between the anode (1) and the hole-transporting layer (2); an electron injection layer between the electron-transporting layer (6) and the cathode (7).

The person skilled in the art knows how suitable materials have to be chosen (for example on the basis of electrochemical studies). Suitable materials for the individual layers are known to those skilled in the art and are disclosed, for example, in WO 00/70655.

In addition, it is possible that some or all of the layers used in the OLED of the invention have been surface-treated in order to increase the efficiency of charge carrier transport. The selection of the materials for each of the layers mentioned is preferably determined so as to obtain an OLED having a high efficiency and lifetime.

The OLEDs of the invention can be produced by methods known to those skilled in the art. In general, the OLED of the invention is produced by successive vapor deposition of the individual layers onto a suitable substrate. Suitable substrates are, for example, glass, inorganic semiconductors or polymer films. Vapor deposition can be accomplished using customary techniques such as thermal evaporation, chemical vapor deposition (CVD), physical vapor deposition (PVD) and others. In an alternative process, the organic layers of the OLED can be applied from solutions or dispersions in suitable solvents, employing coating techniques known to those skilled in the art.

In general, the various layers have the following thicknesses: anode (1) 50 to 500 nm, preferably 100 to 200 nm; hole-conducting layer (2) 5 to 100 nm, preferably 20 to 80 nm, blocking layer for electrons/excitons (3) 2 to 100 nm, preferably 5 to 50 nm, light-emitting layer (4) 1 to 100 nm, preferably 10 to 80 nm, blocking layer for holes/excitons (5) 2 to 100 nm, preferably 5 to 50 nm, electron-conducting layer (6) 5 to 100 nm, preferably 20 to 80 nm, cathode (7) 20 to 1000 nm, preferably 30 to 500 nm. The relative position of the recombination zone of holes and electrons in the OLED of the invention in relation to the cathode and hence the emission spectrum of the OLED can be affected by factors including the relative thickness of each layer. This means that the thickness of the electron transport layer should preferably be chosen such that the position of the recombination zone is matched to the optical resonator property of the diode and hence to the emission wavelength of the emitter. The ratio of the layer thicknesses of the individual layers in the OLED is dependent on the materials used. The layer thicknesses of any additional layers used are known to those skilled in the art. It is possible that the electron-conducting layer and/or the hole-conducting layer have greater thicknesses than the layer thicknesses specified when they are electrically doped.

According to the invention, the light-emitting layer and/or at least one of the further layers that are optionally present in the OLED of the invention comprises at least one compound as described herein. While the at least one compound as described herein is present as emitter material and/or matrix material in the light-emitting layer, the at least one compound as described herein may be used in the at least one further layer of the OLED of the invention, in each case alone or together with at least one of the further materials mentioned above that are suitable for the corresponding layers. It is likewise possible that the light-emitting layer comprises one or more further emitter and/or matrix materials as well as the compound as described herein.

The efficiency of the OLEDs of the invention can be improved, for example by optimizing the individual layers. For example, it is possible to use high-efficiency cathodes such as Ca or Ba, optionally in combination with an intermediate layer of LiF. Shaped substrates and novel hole-transporting materials that bring about a reduction in the operating voltage or an increase in the quantum efficiency are likewise usable in the OLEDs of the invention. In addition, additional layers may be present in the OLEDs in order to adjust the energy level of the various layers and in order to facilitate electroluminescence.

The OLEDs of the invention can be used in all devices in which electroluminescence is useful. Suitable devices are preferably selected from stationary and mobile display screens and lighting units. Stationary display screens are, far example, screens of computers, televisions, screens in printers, kitchen appliances and advertising panels, lighting units and information panels. Mobile display screens are, for example, screens in cellphones, laptops, digital cameras, motor vehicles, and destination displays on buses and trains.

In addition, it is possible to use the compounds as described herein in various embodiments in OLEDs with inverse structure. The construction of inverse OLEDs and the materials that are typically used therein are known to those skilled in the art.

All the documents cited are incorporated herein in their entirety by reference. Further embodiments can be found in the examples which follow, but the invention is not restricted thereto.

It will be apparent and is the intention that all embodiments disclosed herein in connection with the compounds described are equally applicable to the uses and methods described, and vice versa. Embodiments of this kind are therefore likewise covered by the scope of the present invention.

### EXAMPLES

I.) Synthesis Examples

Bromination of Phenothiazine:

5.0 g (0.025 mol) of phenothiazine were suspended in 200 mL of glacial acetic acid, and the mixture was freed of oxygen by introducing Ar through a cannula for 20 minutes. Then 3.3 mL of Br₂ (0.063 mol) in 200 mL of glacial acetic acid were slowly added dropwise over one hour and the dark-colored mixture was stirred for 16 h. After addition of 6.3 g (0.050 mol) of Na₂SO₃, the reaction mixture solidified. Adding a little water gave rise to a violet mass of high viscosity that turned a lighter color within 3 h. After addition of 4.1 g (0.062 mol) of KOH while cooling with ice, the mixture was poured onto 500 mL of ice-water. The greenish precipitate was filtered off with suction and washed with a little cold 2-propanol. The precipitate was subjected to hot digestion and dissolution five times in 200 mL each time of 2-propanol. Needles precipitated out of 2-propanol in the first fraction, and fine flakes in the subsequent fractions. After the crystals had been filtered off with suction and the mother liquor had been concentrated, 7.54 g (85%) of 3,7-dibromophenothiazine were obtained in the form of green crystals.

¹H NMR (500 MHz, d-DMSO): δ (ppm) 6.58 (d, J=8.1 Hz, 2H), 7.10-7.15 (m, 4H), 8.84 (s, 1H).

¹³C NMR (125 MHz, d-DMSO): δ 112.7; 116.0; 118.2; 128.1; 130.3; 140.8.

MS (FAB+) m/z (%): 356.9 (100, M+).

### N-Alkylation of 3,7-dibromophenothiazine

10 g (0.028 mol) of 3,7-dibromophenothiazine and 5.24 g (0.033 mol) of ethyl iodide were dissolved in 100 mL of DMF. 3.36 g (0.14 mol) of NaH (5.6 g of a 60% dispersion in mineral oil) were added stepwise and the mixture was stirred at room temperature overnight. The reaction mixture was poured into ice-water and filtered. The solids obtained were dissolved in ethyl acetate and the solution was washed with saturated sodium chloride solution and dried over magnesium sulfate. After the solvent had been removed by rotary evaporation under reduced pressure, 9.9 g (92%) of the waxy material 3,7-dibromo-10-ethylphenothiazine were obtained.

¹H NMR (500 MHz, DMSO) δ 1.24 (t, J=6.9 Hz, 3H), 3.85 (q, J=6.9 Hz, 2H), 6.93 (d, J=8.6 Hz, 2H), 7.32-7.36 (m, 4H).

¹³C NMR (125 MHz, d-DMSO): δ 12.3; 41.33; 114; 117.2; 124.8; 128.9; 130.4; 143.4.

### Oxidation of 3,7-dibromo-10-ethylphenothiazine to 3,7-dibromo-10-ethylphenothiazine 5,5-dioxide

2 g (7.8 mmol) of 3,7-dibromo-10-ethylphenothiazine were dissolved in 40 mL of dichloromethane, and 5.4 g (31.3 mmol) of 3-chloroperbenzoic acid were added stepwise. The reaction mixture was stirred at room temperature overnight. The precipitated white solids were washed with dichloromethane. The filtrate and the wash solution were concentrated to dryness under reduced pressure. The white crystalline solids obtained were washed with methanol in order to remove the remaining 3-chloroperbenzoic acid. After drying, 1.25 g (38%) of the white crystalline material 3,7-dibromo-10-ethylphenothiazine 5,5-dioxide were obtained.

¹H NMR (500 MHz, DMSO) δ (ppm) 8.14 (d, J=2.4 Hz, 2H), 7.64 (dd, J=9 Hz, J=2.4 Hz, 2H), 7.19 (t, J=4.6 Hz, 2H), 4.16 (q, J=7.1 Hz, 2H), 1.47 (t, J=7.1 Hz, 3H).

¹³C NMR (125 MHz, d-DMSO): δ (ppm) 12.41; 43.49; 114.41; 117.59; 125.15; 126.28; 136.28; 139.19.

MS (ESI) m/z : 356.9 ([M⁺]+1).

Synthesis of the Nonoxidized Phenothiazine Trimer

Into a two-neck flask containing 3,7-dibromo-10-ethylphenothiazine (2.51 g, 6.55 mmol), phenothiazine (2.87 g, 14.42 mmol), Pd₂(dba)₃ (0.18 g, 0.197 mmol) and sodium tert-butoxide (0.53 g, 5.5 mmol) were introduced a solution of tri-tert-butylphosphine in o-xylene [1 M] (0.32 mL) and 40 mL of 1,4-dioxane under an argon atmosphere. The mixture was stirred at 95° C. for 12 hours. After cooling, the solvent was removed and the remaining solids were dispersed in an ultrasound waterbath and filtered. The precipitated material obtained was washed with methanol and diethyl ether. The solids obtained were purified via column chromatography with dichloromethane as eluent. 2.84 g (69%) of yellowish crystals were obtained.

¹H NMR (500 MHz, DMSO) δ 1.43 (t, J=6.9 Hz, 3H), 4.07 (q, J=6.8 Hz, 2H), 5.76 (s, 2H), 6.26 (d, J=8.2 Hz, 2H), 6.85 (t, J=7.4 Hz, 4H), 6.96 (t, J=7.2 Hz, 4H), 7.05 (d, J=7.5 Hz, 4H), 7.32-7.23 (m, 6H).

¹³C NMR (125 MHz, d-DMSO): δ 12.4; 54.9; 115; 117.3; 119.1; 122.7; 124.6; 126.6; 127.4; 128.9; 130.0; 133.4; 134.7; 143.6.

MS (ESI) m/z : 622 ([M⁺]+1).

Synthesis of the Phenothiazine Dioxide Trimer 2 g (3.22 mmol) of the above-described phenothiazine trimer dissolved in 50 mL of N-methyl-2-pyrrolidone (NMP) and 11.1 g (64 mmol) of 3-chloroperbenzoic acid were introduced stepwise into the reaction vessel. The reaction mixture was stirred at 90° C. overnight. The white precipitate of the product formed in the reaction was filtered and washed in methanol and acetone. After drying, 1.67 g (73%) of white crystalline phenothiazine dioxide trimer were obtained.

¹H NMR (300 MHz, CDCl₃) δ 1.71 (t, J=7 Hz, 3H), 4.46 (q, J=7 Hz, 2H), 6.62 (d, J=8.4 Hz, 4H), 7.28-7.20 (m, 4H), 7.38 (ddd, J=8.7, 7.3, 1.6 Hz, 4H), 7.76-7.62 (m, 4H), 8.14 (dd, J=7.9, 1.5 Hz, 4H), 8.23 (d, J=2.4 Hz, 2H).

¹³C NMR (75 MHz, d-DMSO): δ 17.7; 49.4; 117.0; 119.4; 122.7; 123.4; 123.7; 126.5; 133.0; 133.2; 135.9; 140.6; 140.8.

MS (ESI) m/z : 718 ([M^{α}]+1).

Synthesis of the partially oxidized phenothiazine-phenothiazinealkyl dioxide-phenothiazine trimer 3,7-Dibromo-10-ethylphenothiazine 5,5-dioxide (2 g, 4.8 mmol), phenothiazine (2.1 g, 10.5 mmol), Pd₂(dba)₃ (0.132 mg, 0.144 mmol) and sodium tert-butoxide (1.06 g, 11 mmol) were added to a solution of tri-tert-butylphosphine [1 M] (0.24 mL) and 45 mL of dioxane under an argon atmosphere. The mixture was stirred at 95° C. for 24 hours. The reaction mixture was cooled and added to water. After an ultrasound treatment, the white precipitate was filtered and washed with methanol. This material was purified by column chromatography in trichloromethane as eluent. After drying, 1.62 g (52%) of a white powder were obtained.

¹H NMR (500 MHz, DMSO) δ (ppm) 8.01 (d, J=9.2 Hz, 2H), 7.96 (d, J=2.6 Hz, 2H), 7.88 (dd, J=9.1, 2.6 Hz, 2H), 7.15 (dd, J=7.6, 1.5 Hz, 4H), 7.03-6.99 (m, 4H), 6.93 (td, J=7.5, 1.2 Hz, 4H), 6.31 (dd, J=8.2, 1.1 Hz, 4H), 4.53 (q, J=6.9 Hz, 2H), 1.55 (t, J=7.0 Hz, 3H).

¹³C NMR (125 MHz, d-DMSO): δ (ppm) 12.22; 43.14; 116.93; 120.09; 120.68; 123.22; 123.37; 124.18; 127.04; 127.55; 134.7; 135.75; 139.13; 143.34.

MS (ESI) m/z : 654 ([M⁺]+1).

### Methyl 2-aminobenzoate

In a round-bottom flask, 2-(phenylamino)benzoic acid (20 g, 0.146 mol) was dissolved in methanol and stirred in an ice bath for 10 minutes. At 0° C., SOCl₂ (42 mL, 0.584 mol) was cautiously added dropwise and the mixture was stirred under reflux (90° C.) for 12 hours. Thereafter, the reaction mixture was washed with distilled water and the product was extracted with ethyl acetate. The organic phase was dried with MgSO₄ and concentrated on a rotary evaporator. The product was purified by column chromatography with ethyl acetate. The yield was 17.5 g of a partly crystalline substance.

¹H NMR (300 MHz, CDCl₃) δ (ppm): 7.79-7.75 (m, 1H), 7.21-7.14 (m, 1H), 6.59-6.52 (m, 2H), 5.64 (s, 1H), 3.78 (s, 3H).

FT-IR (ATR), v (cm⁻¹): 3481, 3371, 1691, 1615, 1578, 1455, 1435, 1294, 1245, 750.

### Methyl 2-(phenylamino)benzoate

Methyl 2-aminobenzoate (15 g, 0.101 mol), bromobenzene (15.6 g, 0.101 mol), Pd(OAc)₂, (0.45 g, 2.02 mmol), K₂O0₃ (27 g, 0.202 mol), BINAP (1.25 g, 2.02 mmol) and 50 mL of toluene were introduced into a two-neck round-bottom flask under argon. The mixture was stirred at 70° C. overnight. After cooling, the inorganic residues were filtered off and the solvent was concentrated by evaporation. The product was purified by column chromatography with hexane/ethyl acetate (10/1) and then dried. The yield was 10.42 g (46%) of a white powder.

¹H NMR (500 MHz, CDC1₃) δ (ppm) 9.5 (s, 1H), 7.98 (dd, J=8 Hz, J=1.3 Hz, 1H), 7.38-7.24 (m, 6H), 7.10 (t, J=7.3 Hz, 1H), 6.74 (ddd, J=8.1, 6.9, 1.3 Hz, 1H), 3.91 (s, 3H).

FT-IR (ATR), v (cm⁻¹): 3319, 2949, 1686, 1591, 1515, 1453, 1258, 747.

### 2-(2-Anilinophenyl)propan-2-ol

Methyl 2-(phenylamino)benzoate (5.89 g, 0.026 mol) in 100 mL of pure THF were introduced into a baked-out Schlenk flask under nitrogen and cooled down to 0° C. Also added dropwise were 40 mL (0.12 mol) of a 3M MeMgBr solution in diethyl ether, and the mixture was stirred in a nitrogen atmosphere under reflux for 15 hours. Subsequently, the reaction mixture was mixed with saturated ammonium chloride solution, and the organic phase was removed, washed with aqueous sodium chloride solution, dried over MgSO₄ and concentrated on a rotary evaporator under reduced pressure. The product obtained was utilized for the subsequent synthesis steps, without additional purification.

FT-IR (ATR), v (cm⁻¹): 3351, 2976, 1589, 1513, 1496, 1454, 1311, 745.

### 9,9-Dimethyl-10H-acridine

6 mL of concentrated sulfuric acid were added to the oily substance obtained beforehand, and the mixture was subsequently stirred at room temperature under nitrogen for one hour. After dilution with water (200 mL), aqueous ammonia (10% (v/v)) was added up to pH 7. This mixture was poured onto water (50 mL) and extracted with ethyl acetate (150 mL). The cleaned organic phase was further washed with saturated sodium carbonate, sodium chloride solution and water, dried with MgSO₄ and concentrated on a rotary evaporator under reduced pressure. The remaining residue was purified by column chromatography with chloroform/isohexane (1/1). The yield was 3.13 g of yellowish crystals.

¹H NMR (500 MHz, CDC1₃) δ (ppm) 7.38-7.24 (m, 6H), 7.10 (t, J=7.3 Hz, 1H), 6.74 (ddd, J=8.1, 6.9, 1.3 Hz, 1 H), 1.6 (s, 3H).

3,7-Bis(9,9-dimethylacridin-10-yl)-10-ethylphenothiazine 3,7-Dibromo-10-ethylphenothiazine (0.997 g, 2.6 mmol), 9,9-dimethyl-10H-acridine (1.09 g, 5.21 mmol), Pd₂(dba)₃ (0.071 g, 0.078 mmol), sodium t-butoxide (0.575 g) were added to a two-neck round-bottom flask. The flask was filled with argon, and a 1M solution of tri-t-butylphosphine in o-xylene (0.12 mL) and 20 mL of dioxane were added. The mixture was subsequently stirred at 95° C. for 24 hours. After cooling, the solvent was removed and the solid substance remaining was carefully dispersed in water in an ultrasound bath and filtered off. The precipitate obtained was further washed with methanol and diethyl ether. The solid product thus obtained was then purified via column chromatography with dichloromethane. The yield was 0.7 g (42%) of yellowish crystals.

MS (ESI) m/z : 642 ([M⁺]+1).

### 3,7-Bis(9,9-dimethylacridin-10-yl)-10-ethylphenothiazine 5,5-dioxide

3,7-Dibromo-10-ethylphenothiazine 5,5-dioxide (1.36 g, 3.25 mmol), 9,9-dimethyl-10H-acridine (1.5 g, 7.17 mmol), Pd₂(dba)₃ (0.09 g, 0.097 mmol) and sodium t-butoxide (0.72 g, 7.45 mmol) were added to a two-neck round-bottom flask. The flask was filled with argon, and then a 1M solution of tri-t-butylphosphine in o-xylene (0.16 mL) and 40 mL of dioxane were added. The mixture was then stirred at 95° C. for 12 hours. After cooling, the solvent was removed and the solid substance remaining was carefully dispersed in water in an ultrasound bath and filtered off. The precipitate obtained was then further washed with methanol and acetone. The crude product was dried and gave a yield of 1.44 g (66%) of white crystals.

¹H NMR (500 MHz, DMF) δ (ppm) 8.25 (d, J=9.0 Hz, 2H), 8.07 (d, J=2.4 Hz, 2H), 7.94 (dd, J=9.0, 2.4 Hz, 2H), 7.58 (d, J=8.5 Hz, 4H), 7.09 7.03 (m, 4H), 6.99 (t, J=7.5 Hz, 4H), 6.30 (d, J=7.2 Hz, 4H), 4.74 (q, J=7.3 Hz, 2H), 0.87 (t, J=7 Hz, 3H).

II.) Spectroscopic Studies

As an example of phosphorescence spectra, FIG. 1 shows the phosphorescence spectra of the three substances phenothiazine 5,5-dioxide (PTO, recorded at a temperature of T =293 K), phenothiazine (PT, at T =80 K), and 9,9-dimethyl-9,10-dihydroacridine (DMAC, at T =80 K), recorded on thin polystyrene films, each of which contains 2% of these materials. Compared to solutions in less polar solvents, these phosphorescence spectra are already somewhat red-shifted. The vertical transition energies Eᵥₑᵣₜ(T₁→S₀) as defined herein are respectively 2,25 eV (PTO), 2.21 eV (PT), and 2.21 eV (DMAC). The tangent shown for PT at half the height of the flank at short wavelengths gives an interpolated minimum of the spectrum at 466 nm or 2,86 eV.

In the case of line shapes similar to those in FIG. 1, the threshold of 2.2 eV for the vertical transition energy Eᵥₑᵣₜ(T₁→S₀) thus corresponds to the onset of phosphorescence on the upper energetic flank at about 2,65 eV, or at a higher energy by 0.45 eV. Other possible definitions of the phosphorescence energy such as the maximum of the wavelength-dependent intensity l(A) or the maximum of the energy-dependent intensity I(E) lead to energy values between Eᵥₑᵣₜ and the edge of onset at high energy (short wavelength).

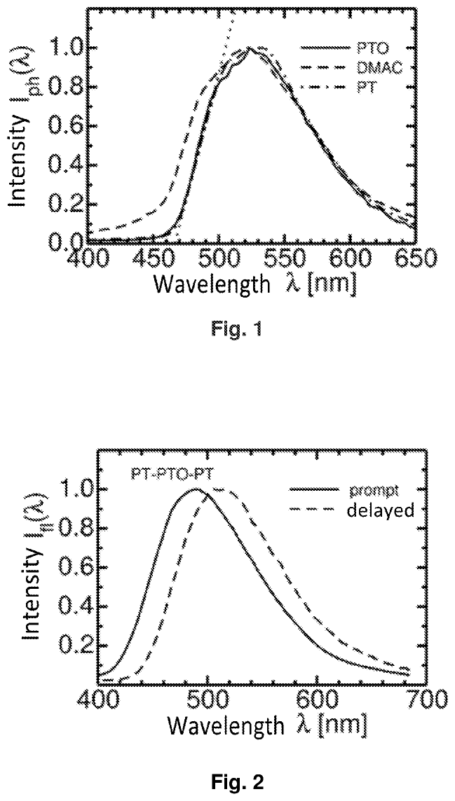

The spectra of selected examples from the substance class as defined herein are shown in FIGS. 2 to 5.

FIG. 2 shows the prompt and delayed fluorescence of a trimer composed of a central phenothiazine 5,5-dioxide (PTO), and two phenothiazine (PT) groups, abbreviated to PT-PTO-PT, embedded in a concentration of 2% in a thin film of polystyrene. Excitation here was effected by means of a short light pulse of duration 120 ps, at a wavelength of 335 nm. The prompt fluorescence was integrated from a delay of 2.25 ns onward over a period of 1.7 ns, and the delayed fluorescence from a delay of 1 μs onward over a period of 80 μs.

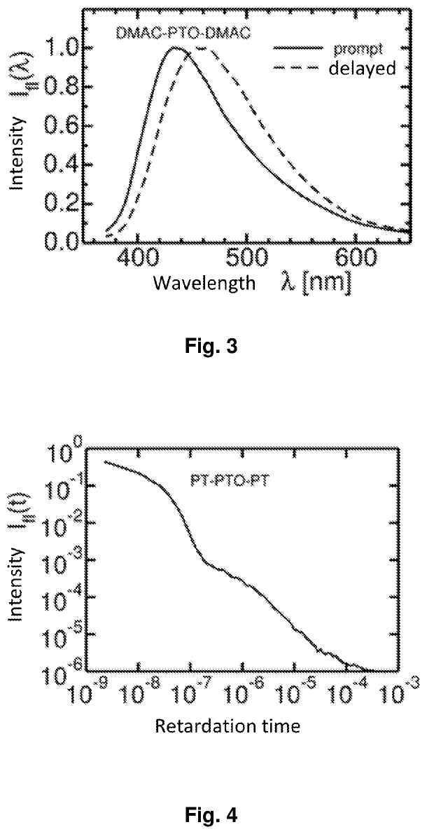

FIG. 3 shows the prompt and delayed fluorescence of a trimer composed of a central phenothiazine 5,5-dioxide, and two dimethylacridine (DMAC) groups, abbreviated to DMAC-PTO-DMAC, embedded in a concentration of 2% in a thin film of polystyrene. Excitation conditions and time windows as for FIG. 2.

FIG. 4 shows the evolution against time of the fluorescence intensity of the PT-PTO-PT trimer, embedded in a concentration of 2% in a thin film of polystyrene. The different dynamics over time of the prompt and delayed fluorescence demonstrate the involvement of time-delayed thermally activated fluorescence (TADF). Excitation conditions as for FIG. 2.

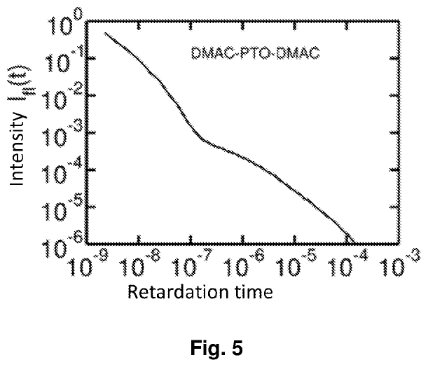

FIG. 5 shows the evolution against time of the fluorescence intensity of the DMAC-PTO-DMAC trimer, embedded in a concentration of 2% in a thin film of polystyrene. The different dynamics over time of the prompt and delayed fluorescence demonstrate the involvement of time-delayed thermally activated fluorescence (TADF). Excitation conditions as for FIG. 2.

1. A compound comprising at least one donor group and at least one acceptor group, in which the vertical transition energy of the lowest excited triplet back to the electronic ground state both for the individual corresponding donor molecule and for the individual corresponding acceptor molecule is at least 2.2 eV, and wherein the dihedral angle between the at least one donor group and the at least one acceptor group is at least 70°.

2. The compound as claimed in claim 1, wherein the at least one donor group and/or acceptor group is a compound of the formula (1) or a compound of the formula (II): 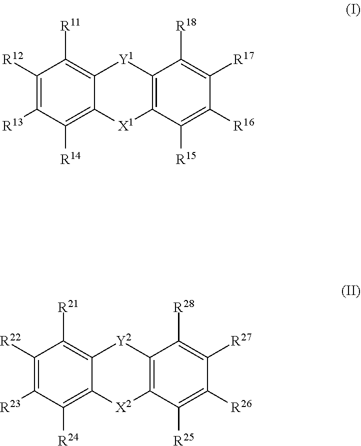
where, in the compounds of the formula (I),
R¹¹-R¹⁸ are each independently selected from the group consisting of hydrogen, C₁-C₂₀ alkyl, aryl and bromine, or R¹¹, R¹⁴, R¹⁵ and R¹⁸ are as defined above and R¹³ and/or R¹⁶ or R¹² and/or R¹⁷ denotes 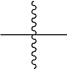
which represents the point of attachment to another part of the molecule;
Y¹ is selected from the group consisting of N—R¹⁹, P(O)R¹⁹ and P(O)OR¹⁹ and X¹ is selected from the group consisting of P(O)R¹⁹, P(O)OR¹⁹, Si(C₁-C₂₀ alkyl)₂, Si(aryl)₂ and SO₂;
where R¹⁹ is in each case independently selected from the group consisting of hydrogen, C₁-C₂₀ alkyl and aryl, or R¹⁹ denotes 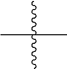
which represents the point of attachment to another part of the molecule;
and where, in the compounds of the formula (II),
R²¹-R²⁸ are each independently selected from the group consisting of hydrogen, C₁-C₂₀ alkyl, aryl and bromine, or R21, _{R}24_{, R}25 an_{d} are as defined above and R²³ and/or R²⁶ R²⁸ or R²² and/or R²⁷ denotes 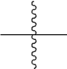
which represents the point of attachment to another part of the molecule;
Y² is selected from the group consisting of P(O)R²⁹, P(O)OR²⁹, Si(C₁-C₂₀ alkyl)₂, Si(aryl)₂ and SO₂;
X² is selected from the group consisting of P(O)R²⁹, P(O)OR²⁹, Si(Cᵢ-C₂₀ alkyl)₂, Si(aryl)₂ and SO₂;
where R²⁹ is in each case independently selected from the group consisting of hydrogen, C₁-C₂₀ alkyl and aryl, or R²⁹ denotes 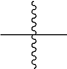
which represents the point of attachment to another part of the molecule.

3. The compound as claimed in claim 2, wherein R¹⁹ and/or R²⁹ is/are each independently selected from the group consisting of hydrogen, C₁-C₂₀ alkyl and phenyl, or R¹⁹ and/or R²⁹ denote(s) 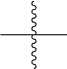
which represents the point of attachment to another part of the molecule.

4. The compound as claimed in claim 1, wherein the compound, in addition to the at least one donor molecule and the at least one acceptor molecule, comprises at least one further donor molecule or acceptor molecule in which the vertical transition energy of the lowest excited triplet is at least 2.2 eV.

5. The compound as claimed in claim 4, wherein the at least one further donor group or acceptor group is a compound of the formula (I) or (11) as defined in claim 3.

6. The compound as claimed in claim 1, wherein the compound is a compound of the formula (III) or a compound of the formula (IV):
A-B-A (III)
A-B (IV)
wherein, in the compounds of the formula (III) or (IV),
components A and B are each either a donor group or an acceptor group, where at least one A or B is, preferably all A and more preferably all A and B are, a compound having a vertical transition energy of the lowest excited triplet back to the electronic ground state of at least 2.2 eV, and wherein the dihedral angle between at least one donor group and at least one acceptor group, preferably between (each) A and B, is at least 70°.

7. The compound as claimed in claim 6, wherein component A is in each case independently a compound of the formula (I) as defined in claim 3 which is bonded to component B via R¹⁹ in each case.

8. The compound as claimed in claim 7, wherein component B is a compound having a vertical transition energy of the lowest excited triplet back to the electronic ground state of at least 2.2 eV, especially selected from carbazole and carbazole-containing compounds, dihydroacridine, dimethylacridine, phenothiazine and phenoxazine.

9. The compound as claimed in claim 6, wherein component B is a compound of the formula (I) or of the formula (H) as defined in claim 3, each of which is bonded to component(s) A via (a) R¹³ and/or R¹⁶ or (b) R¹² and/or R¹⁷ or (c) R²³ and/or R²⁶ or (d) R²² and/or R²⁷.

10. The compound as claimed in claim 9, wherein each component A is independently selected from compounds having a vertical transition energy of the lowest excited triplet back to the electronic ground state of at least 2.2 eV, especially selected from carbazole and carbazole-containing compounds, dihydroacridine, dimethylacridine, phenothiazine and phenoxazine.

11. The compound as claimed in claim6, wherein the compound is selected from the group of compounds of the formulae (1)-(14):

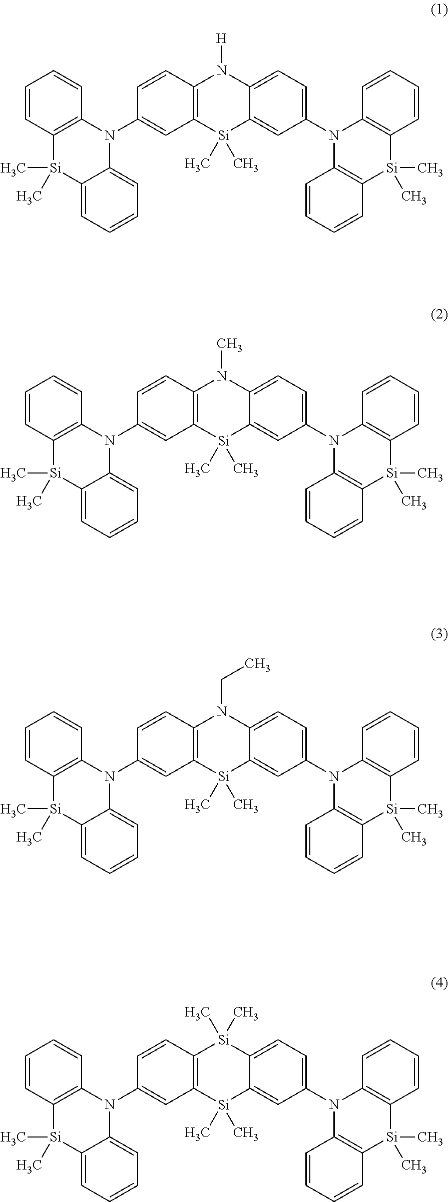

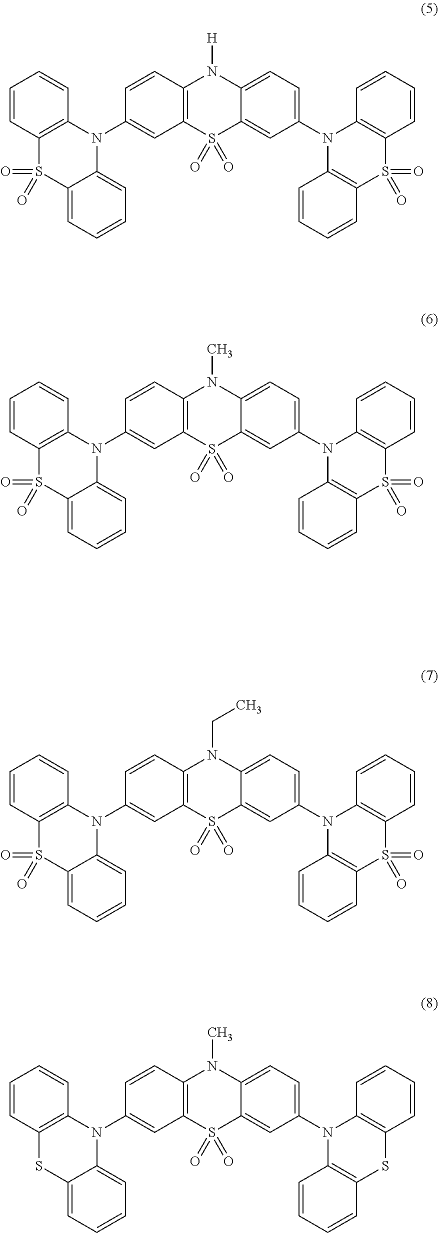 [SMILES: O=S1(=O)c2cc(N3c4ccccc4S(=O)(=O)c4ccccc43)ccc2Nc2ccc(N3c4ccccc4S(=O)(=O)c4ccccc43)cc21 ; CN1c2ccc(N3c4ccccc4S(=O)(=O)c4ccccc43)cc2S(=O)(=O)c2cc(N3c4ccccc4S(=O)(=O)c4ccccc43)ccc21 ; CCN1c2ccc(N3c4ccccc4S(=O)(=O)c4ccccc43)cc2S(=O)(=O)c2cc(N3c4ccccc4S(=O)(=O)c4ccccc43)ccc21 ; CN1c2ccc(N3c4ccccc4Sc4ccccc43)cc2S(=O)(=O)c2cc(N3c4ccccc4Sc4ccccc43)ccc21]

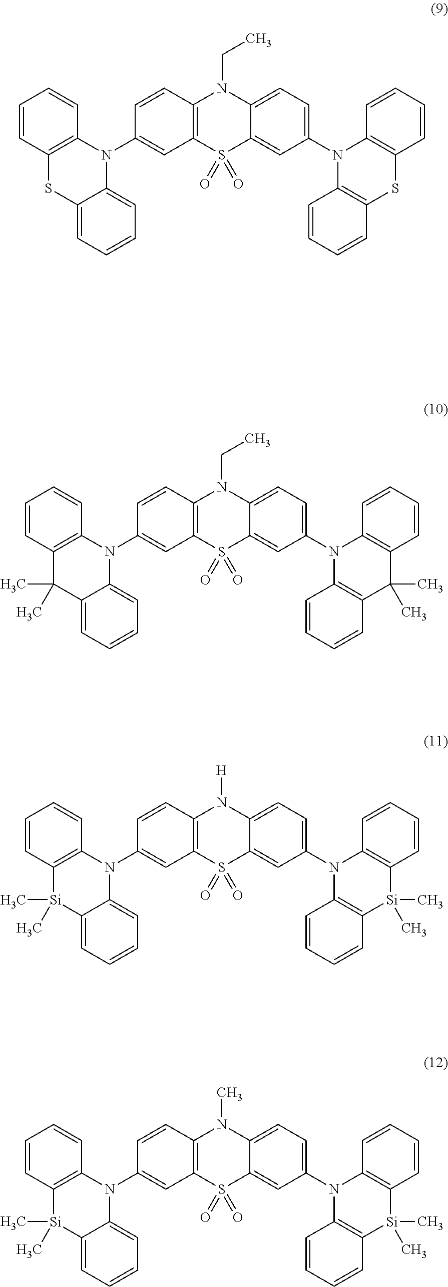

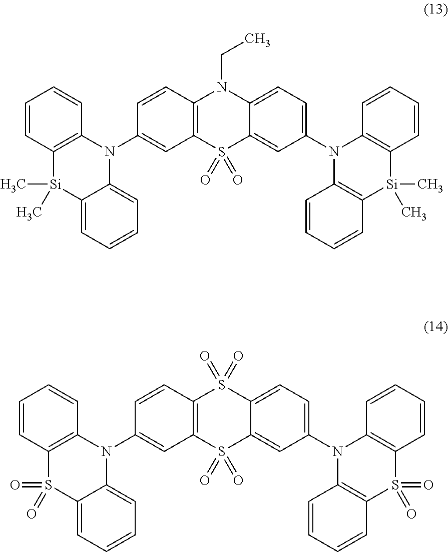

12. The compound as claimed in claim1, wherein the compound exhibits thermally activatable delayed fluorescence.

13. The use of a compound as claimed in claim1 in an optoelectronic component, for example an organic electroluminescent device (OLED), an organic integrated circuit (O-IC), an organic field-effect transistor (O-FET), an organic thin--film transistor (O-TFT), an organic light-emitting transistor (O-LET), an organic solar cell (O-SC), an organic optical detector, an organic photoreceptor, an organic field-quench device (O-FQD), a light-emitting electrochemical cell (LEC) or an organic laser diode (O-laser) as emitter or matrix material, preferably as emitter.
